(19) United States
(12) Patent Application Publication
(10) Pub. No.: US 2002/0142993 A1
(43) Pub. Date: Oct. 3, 2002

(54) Pharmaceutically acceptable salts of bicyclic compounds

(75) Inventors: Om Reddy Gaddam, Hyderabad (IN); Chandra Sekhar Batchu, Hyderabad (IN); Ramabhadra Sarma Mamillapalli, Hyderabad (IN); Prasad Sivarama Addanki, Hyderabad (IN)

Correspondence Address: LADAS & PARRY, 26 WEST 61ST STREET, NEW YORK, NY, 10023, US

(73) Assignee: DR. REDDY'S LABORATORIES LTD.

(21) Appl. No.: 09/975,741

(22) Filed: Oct. 10, 2001

Related U.S. Application Data
(60) Provisional application No. 60/266,996, filed on Feb. 5, 2001.

(30) Foreign Application Priority Data
Oct. 12, 2000 (IN) 863/MAS/2000

(51) Int. Cl. A61K 31/7008; A61K 31/5415; A61K 31/538; C07H 5/06; C07D 279/16; C07D 265/36

(52) U.S. Cl. 514/62; 514/224.2; 514/230.5; 544/105; 544/51; 536/18.7

(57) ABSTRACT

The present invention relates to pharmaceutically acceptable salts of compound of the general formula (I), their derivatives, their analogs, their tautomeric forms, their stereoisomers, their polymorphs, their pharmaceutically acceptable solvates and pharmaceutically acceptable compositions containing them.

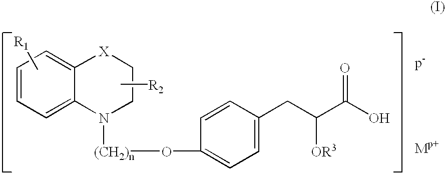

### FIELD OF THE INVENTION

The present invention relates to pharmaceutically acceptable salts of compound of the general formula (I), their derivatives, their analogs, their tautomeric forms, their stereoisomers, their polymorphs, their pharmaceutically acceptable solvates and pharmaceutically acceptable compositions containing them.

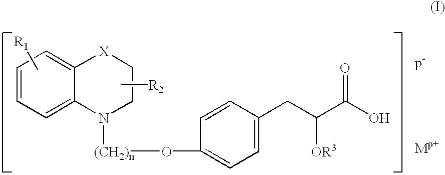

The present invention also relates to a process for the preparation of the above said pharmaceutically acceptable salts, their derivatives, their analogs, their tautomeric forms, their stereoisomers, their polymorphs, pharmaceutically acceptable solvates, and pharmaceutical compositions containing them.

The compounds of the present invention lower plasma glucose, triglycerides, lower total cholesterol (TC) and increase high density lipoprotein (HDL) and decrease low density lipoprotein (LDL), which have a beneficial effect on coronary heart disease and atherosclerosis.

The compounds of general formula (I) are useful in reducing body weight and for the treatment and/or prophylaxis of diseases such as atherosclerosis, stroke, peripheral vascular diseases and related disorders. These compounds are useful for the treatment of hyperlipemia, hyperglycemia, hyper-cholesterolemia, lowering of atherogenic lipoproteins, VLDL (very low density lipoprotein) and LDL. The compounds of the present invention can be used for the treatment of certain renal diseases including glomerulonephritis, glomerulosclerosis, nephrotic syndrome, hypertensive nephrosclerosis and nephropathy. The compounds of general formula (I) are also useful for the treatment and/or prophylaxis of leptin resistance, impaired glucose tolerance, disorders related to syndrome X such as hypertension, obesity, insulin resistance, coronary heart disease and other cardiovascular disorders. These compounds may also be useful as aldose reductase inhibitors, for improving cognitive functions in dementia, treating diabetic complications, disorders related to endothelial cell activation, psoriasis, polycystic ovarian syndrome (PCOS), inflammatory bowel diseases, osteoporosis, myotonic dystrophy, pancreatitis, arteriosclerosis, retinopathy, xanthoma, eating disorders, inflammation and for the treatment of cancer. The compounds of the present invention are also useful in the treatment and/or prophylaxis of the above said diseases in combination/concomittant with one or more HMG CoA reductase inhibitors, hypolipidemic/hypolipoproteinemic agents such as fibric acid derivatives, nicotinic acid, cholestyramine, colestipol and probucol.

### BACKGROUND OF THE INVENTION

Atherosclerosis and other peripheral vascular diseases effect the quality of life of millions of people. Therefore, considerable attention has been directed towards understanding the etiology of hypercholesterolemia and hyperlipidemia and development of effective therapeutic strategies.

Hypercholesterolemia has been defined as plasma cholesterol level that exceeds arbitrarily defined value called “normal” level. Recently, it has been accepted that “ideal” plasma levels of cholesterol are much below the “normal” level of cholesterol in the general population and the risk of coronary artery disease (CAD) increases as cholesterol level rises above the “optimum” (or “ideal”) value. There is clearly a definite cause and effect-relationship between hypercholesterolemia and CAD, particularly for individuals with multiple risk factors. Most of the cholesterol is present in the esterified forms with various lipoproteins such as Low density lipoprotein (LDL), Intermediate density lipoprotein (IDL), High density lipoprotein (HDL) and partially as Very low density lipoprotein (VLDL). Studies clearly indicate that there is an inverse correlationship between CAD and atherosclerosis with serum HDL-cholesterol concentrations, (Stampfer et al., N. Engl. J. Med., 325 (1991), 373-381) and the risk of CAD increases with increasing levels of LDL and VLDL.

In CAD, generally “fatty streaks” in carotid, coronary and cerebral arteries, are found which are primarily free and esterified cholesterol. Miller et al., (Br. Med. J., 282 (1981), 1741-1744) have shown that increase in HDL-particles may decrease the number of sites of stenosis in coronary arteries of human, and high level of HDL-cholesterol may protect against the progression of atherosclerosis. Picardo et al., Arteriosclerosis 6 (1986) 434-441 have shown by in vitro experiment that HDL is capable of removing cholesterol from cells. They suggest that HDL may deplete tissues of excess free cholesterol and transfer it to liver (Macikinnon et al., J. Biol. chem. 261 (1986), 2548-2552). Therefore, agents that increase HDL cholesterol would have therapeutic significance for the treatment of hypercholesterolemia and coronary heart diseases (CHD).

Obesity is a disease highly prevalent in affluent societies and in the developing world and is a major cause of morbidity and mortality. It is a state of excess body fat accumulation. The causes of obesity are unclear. It is believed to be of genetic origin or promoted by an interaction between the genotype and environment. Irrespective of the cause, the result is fat deposition due to imbalance between the energy intake versus energy expenditure. Dieting, exercise and appetite suppression have been a part of obesity treatment. There is a need for efficient therapy to fight this disease since it may lead to coronary heart disease, diabetes, stroke, hyperlipidemia, gout, osteoarthritis, reduced fertility and many other psychological and social problems.

Diabetes and insulin resistance is yet another disease which severely effects the quality of large population in the world. Insulin resistance is the diminished ability of insulin to exert its biological action across a broad range of concentrations. In insulin resistance, the body secretes abnormally high amounts of insulin to compensate for this defect; failing which, the plasma glucose concentration inevitably rises and develops into diabetes. Among the developed countries, diabetes mellitus is a common problem and is associated with a variety of abnormalities including obesity, hypertension, hyperlipidemia (J. Clin. Invest., 75 (1985) 809-817; N. Engl. J. Med 317 (1987) 350-357; J. Clin. Endocrinol. Metab., 66 (1988) 580-583; J. Clin. Invest., 68 (1975) 957-969) and other renal complications (patent publication No. WO 95/21608). It is now increasingly being recognized that insulin resistance and relative hyperinsulinemia have a contributory role in obesity, hypertension, atherosclerosis and type 2 diabetes mellitus. The association of insulin resistance with obesity, hypertension and angina has been described as a syndrome having insulin resistance as the central pathogenic link-Syndrome-X.

Hyperlipidemia is the primary cause for cardiovascular (CVD) and other peripheral vascular diseases. High risk of CVD is related to the higher LDL (Low Density Lipoprotein) and VLDL (Very Low Density Lipoprotein) seen in hyperlipidemia. Patients having glucose intolerance/insulin resistance in addition to hyperlipidemia have higher risk of CVD. Numerous studies in the past have shown that lowering of plasma triglycerides and total cholesterol, in particular LDL and VLDL and increasing HDL cholesterol help in preventing cardiovascular diseases.

Peroxisome proliferator activated receptors (PPAR) are members of the nuclear receptor super family. The gamma (γ) isoform of PPAR (PPARγ) has been implicated in regulating differentiation of adipocytes (Endocrinology, 135 (1994) 798-800) and energy homeostasis (Cell, 83 (1995) 803-812), whereas the alpha (α) isoform of PPAR (PPARα) mediates fatty acid oxidation (Trend. Endocrin. Metab., 4 (1993) 291-296) thereby resulting in reduction of circulating free fatty acid in plasma (Current Biol. 5 (1995) 618-621). PPARα agonists have been found useful for the treatment of obesity (WO 97/36579). It has been recently disclosed that compounds which are agonists for both PPARα and PPARγ are suggested to be useful for the treatment of syndrome X (WO 97/25042). Similar effect between the insulin sensitizer (PPARγ agonist) and HMG CoA reductase inhibitor has been observed which may be useful for the treatment of atherosclerosis and xanthoma (EP 0 753 298).

It is known that PPARγ plays an important role in adipocyte differentiation (Cell, 87 (1996) 377-389). Ligand activation of PPAR is sufficient to cause complete terminal differentiation (Cell, 79 (1994) 1147-1156) including cell cycle withdrawal. PPARγ is consistently expressed in certain cells and activation of this nuclear receptor with PPARγ agonists would stimulate the terminal differentiation of adipocyte precursors and cause morphological and molecular changes characteristics of a more differentiated, less malignant state (Molecular Cell, (1998), 465-470; Carcinogenesis, (1998), 1949-53; Proc. Natl. Acad. Sci., 94 (1997) 237-241) and inhibition of expression of prostate cancer tissue (Cancer Research 58 (1998) 3344-3352). This would be useful in the treatment of certain types of cancer, which express PPARγ and could lead to a quite nontoxic chemotherapy.

Leptin resistance is a condition wherein the target cells are unable to respond to leptin signal. This may give rise to obesity due to excess food intake and reduced energy expenditure and cause impaired glucose tolerance, type 2 diabetes, cardiovascular diseases and such other interrelated complications. Kallen et al (Proc. Natl. Acad. Sci. (1996) 93, 5793-5796) have reported that insulin sensitizers which perhaps due to the PPAR agonist expression lower plasma leptin concentrations. However, it has been recently disclosed that compounds having insulin sensitizing property also possess leptin sensitization activity. They lower the circulating plasma leptin concentrations by improving the target cell response to leptin (WO 98/02159).

In our International publication Nos. WO 99/20614 and WO 00/66572 we have disclosed and described the novel compounds of the formula (II),

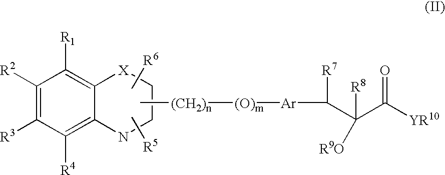

wherein the groups R¹, R², R³, R⁴ and the groups R⁵ and R⁶ when attached to carbon atom, may be same or different and represent hydrogen, halogen, hydroxy, nitro, cyano, formyl or unsubstituted or substituted groups selected from alkyl, cycloalkyl, alkoxy, cycloalkoxy, aryl, aryloxy, aralkyl, aralkoxy, heterocyclyl, heteroaryl, heteroaralkyl, heteroaryloxy, heteroaralkoxy, acyl, acyloxy, amino, acylamino, monoalkylamino, dialkylamino, arylamino, aralkylamino, alkoxycarbonyl, aryloxycarbonyl, aralkoxycarbonyl, alkylthio, alkoxycarbonylamino, aryloxycarbonylamino, aralkoxycarbonylamino, carboxylic acid or its derivatives, or sulfonic acid or its derivatives; one or both of R⁵ and R⁶ may also represent an oxo group when they are attached to carbon atom; R⁵ and R⁶ when attached to nitrogen atom represent hydrogen, hydroxy, formyl or unsubstituted or substituted groups selected from alkyl, cycloalkyl, alkoxy, cycloalkoxy, aryl, aralkyl, heterocyclyl, heteroaryl, heteroaralkyl, acyl, acyloxy, amino, acylamino, monoalkylamino, dialkylamino, arylamino, aralkylamino, aryloxy, aralkoxy, heteroaryloxy, heteroaralkoxy, alkoxycarbonyl, aryloxycarbonyl, aralkoxycarbonyl, alkylthio groups, carboxylic acid derivatives, or sulfonic acid derivatives; X represents a heteroatom selected from oxygen, sulfur or NR¹¹ where R¹¹ represents hydrogen or unsubstituted or substituted groups selected from alkyl, cycloalkyl, aryl, aralkyl, acyl, alkoxycarbonyl, aryloxycarbonyl or aralkoxycarbonyl groups; Ar represents an unsubstituted or substituted divalent single or fused aromatic or heterocyclic group; R⁷ represents hydrogen atom, hydroxy, alkoxy, halogen, lower alkyl, unsubstituted or substituted aralkyl group or forms a bond together with the adjacent group R⁸; R⁸ represents hydrogen, hydroxy, alkoxy, halogen, lower alkyl group, acyl or unsubstituted or substituted aralkyl or R⁸ forms a bond together with R⁷; R⁹ represents hydrogen or unsubstituted or substituted groups selected from alkyl, cycloalkyl, aryl, aralkyl, alkoxycarbonyl, aryloxycarbonyl, alkylaminocarbonyl, arylaminocarbonyl, acyl, heterocyclyl, heteroaryl or heteroaralkyl groups; R¹⁰ represents hydrogen or unsubstituted or substituted groups selected from alkyl, cycloalkyl, aryl, aralkyl, heterocyclyl, heteroaryl or heteroaralkyl groups; Y represents oxygen or NR¹², where R¹² represents hydrogen, alkyl, aryl, hydroxyalkyl, aralkyl, heterocyclyl, heteroaryl or heteroaralkyl groups; R¹⁰ and R¹² together may form a 5 or 6 membered cyclic structure containing carbon atoms, atleast one nitrogen atom and which may optionally contain one or two additional heteroatoms selected from oxygen, sulfur or nitrogen; the linking group represented by —(CH₂)ₙ—(O)ₘ—. may be attached either through a nitrogen atom or a carbon atom; n is an integer ranging from 1-4 and m is an integer 0 or 1. We have also described the processes for preparing the compounds of formula (II).

The pharmaceutically acceptable salts of the general formula (I) have significant formulation and bulk handling advantages in view of the their stability.

### OBJECTIVE OF THE INVENTION

The main objective of the present invention is therefore to provide pharmaceutically acceptable salts of β-aryl-α-oxysubstituted alkyl carboxylic acids of the formula (I), their derivatives, their analogs, their tautomeric forms, their stereoisomers, their polymorphs, their pharmaceutically acceptable solvates and pharmaceutical compositions containing them or their mixtures having good stability and solubility, which can be used for the treatment and/or prophylaxis of diseases related to increased levels of lipids, especially to treat hypertriglyceridemia and to lower free fatty acids, for the treatment and/or prophylaxis of diseases described as Syndrome-X, which include hyperlipidemia, hyperinsulinemia, obesity, insulin resistance, insulin resistance leading to type 2 diabetes and diabetic complications thereof, for the treatment of diseases wherein insulin resistance is the pathophysiological mechanism, for the treatment of hypertension, atherosclerosis and coronary artery diseases with better efficacy, potency and lower toxicity.

Another objective of the present invention is to provide pharmaceutically acceptable salts of β-aryl-α-oxysubstituted alkyl carboxylic acids of the formula (I) and their derivatives, their analogs, their tautomeric forms, their stereoisomers, their polymorphs, their pharmaceutically acceptable solvates and pharmaceutical compositions containing them or their mixtures which may have agonist activity against PPARα and/or PPARγ, and optionally inhibit HMG CoA reductase, in addition to agonist activity against PPARα and/or PPARγ.

Another objective of the present invention is to provide pharmaceutically acceptable salts of β-aryl-α-oxysubstituted alkyl carboxylic acids of the formula (I) and their derivatives, their analogs, their tautomeric forms, their stereoisomers, their polymorphs, their pharmaceutically acceptable solvates and pharmaceutical compositions containing them or their mixtures having enhanced activities, without toxic effect or with reduced toxic effect.

Yet another objective of the present invention is to provide pharmaceutically acceptable salts of the general formula (I) having better stability and physicochemical properties.

Yet another objective of the present invention is to provide a process for the preparation of pharmaceutically salts of β-aryl-α-oxysubstituted alkyl carboxylic acids and their derivatives of the formula (I) as defined above, their analogs, their tautomeric forms, their stereoisomers, their polymorphs, and their pharmaceutically acceptable solvates.

Still another objective of the present invention is to provide pharmaceutical compositions containing compounds of the general formula (I), their analogs, their derivatives, their tautomers, their stereoisomers, their polymorphs, their salts, solvates or their mixtures in combination with suitable carriers, solvents, diluents and other media normally employed in preparing such compositions.

### DETAILED DESCRIPTION OF THE INVENTION

The present invention relates to pharmaceutically acceptable salts having the general formula (I)

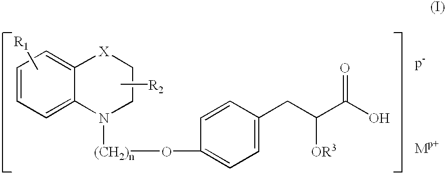

their derivatives, their analogs, their tautomeric forms, their stereoisomers, their polymorphs, wherein R¹ represents hydrogen, halogen atom such as fluorine, chlorine, bromine or iodine; hydroxy, nitro, cyano or lower alkyl group; R²represents hydrogen, lower alkyl or oxo group; X represents a heteroatom selected from oxygen or sulfar; R³ represents hydrogen or lower alkyl group; n is an integer ranging from 1-4; M represents a counter ion (an anion which balances the ionic balance) or a moiety which forms a pharmaceutically acceptable salt; p is an integer ranging from 1 to 2.

The term lower alkyl represents linear or branched (C₁-C₆)alkyl group, such as methyl, ethyl, n-propyl, iso-propyl, n-butyl, iso-butyl, t-butyl, n-pentyl, iso-pentyl, hexyl and the like.

Suitable groups represented by M may be selected from glucamine, methylglucamine, N-octylglucamine, dicyclohexylamine, methyl benzylamine, tris(hydroxymethyl)aminomethane (tromethamine), phenyl glycinol, lysine, aminoguanidine, or aminoguanidine hydrogen carbonate or metformin.

Suitable n is an integer ranging from 1 to 4, preferably n represents an integer 1 or 2.

Particularly useful compounds according to the present invention include:

(±)3-[4-[2-(3,4-Dihydro-2H-1,4-benzoxazin-4-yl)ethoxy]phenyl]-2-ethoxy propanoic acid lysine salt;

(+)3 -[4-[2-(3,4-Dihydro-2H-1,4-benzoxazin-4-yl)ethoxy]phenyl]-2-ethoxy propanoic acid lysine salt;

(−)3-[4-[2-(3,4-Dihydro-2H-1,4-benzothiazin-4-yl)ethoxy]phenyl]-2-ethoxy propanoic acid lysine salt;

(±)3-[4-[2-(3,4-Dihydro-2H-1,4-benzothiazin-4-yl)ethoxy]phenyl]-2-ethoxy propanoic acid lysine salt;

(+)3-[4-[2-(3,4-Dihydro-2H-1,4-benzothiazin-4-yl)ethoxy]phenyl]-2-ethoxy propanoic acid lysine salt;

(−)3-[4-[2-(3,4-Dihydro-2H-1,4-benzoxazin-4-yl)ethoxy]phenyl]-2-ethoxy propanoic acid lysine salt;

(±)3-[4-[2-(3,4-Dihydro-2H-1,4-benzoxazin-4-yl)ethoxy]phenyl]-2-ethoxy propanoic acid dicyclohexylamine salt;

(+)3-[4-[2-(3,4-Dihydro-2H-1,4-benzoxazin-4-yl)ethoxy]phenyl]-2-ethoxy propanoic acid dicyclohexylamine salt;

(−)3-[4-[2-(3,4-Dihydro-2H-1,4-benzoxazin-4-yl)ethoxy]phenyl]-2-ethoxy propanoic acid dicyclohexylamine salt;

(±)3-[4-[2-(3,4-Dihydro-2H-1,4-benzothiazin-4-yl)ethoxy]phenyl]-2-ethoxy propanoic acid dicyclohexylamine salt;

(+)3-[4-[2-(3,4-Dihydro-2H-1,4-benzothiazin-4-yl)ethoxy]phenyl]-2-ethoxy propanoic acid dicyclohexylamine salt;

(−)3-[4-[2-(3,4-Dihydro-2H-1,4-benzothiazin-4-yl)ethoxy]phenyl]-2-ethoxy propanoic acid dicyclohexylamine salt;

(±)3-[4-[2-(3,4-Dihydro-2H-1,4-benzoxazin-4-yl)ethoxy]phenyl]-2-ethoxy propanoic acid metformin salt;

(+)3-[4-[2-(3,4-Dihydro-2H-1,4-benzoxazin-4-yl)ethoxy]phenyl]-2-ethoxy propanoic acid metformin salt;

(−)3-[4-[2-(3,4-Dihydro-2H-1,4-benzoxazin-4-yl)ethoxy]phenyl]-2-ethoxy propanoic acid metformin salt;

(±)3-[4-[2-(3,4-Dihydro-2H-1,4-benzothiazin-4-yl)ethoxy]phenyl]-2-ethoxy propanoic acid metformin salt;

(+)3-[4-[2-(3,4-Dihydro-2H-1,4-benzothiazin-4-yl)ethoxy]phenyl]-2-ethoxy propanoic acid metformin salt;

(−)3-[4-[2-(3,4-Dihydro-2H-1,4-benzothiazin-4-yl)ethoxy]phenyl]-2-ethoxy propanoic acid metformin salt;

(±)3-[4-[2-(3,4-Dihydro-2H-1,4-benzothiazin-4-yl)ethoxy]phenyl]-2-ethoxy propanoic acid phenyl glycinol salt;

(+)3-[4-[2-(3,4-Dihydro-2H-1,4-benzothiazin-4-yl)ethoxy]phenyl]-2-ethoxy propanoic acid phenyl glycinol salt;

(−)3-[4-[2-(3,4-Dihydro-2H-1,⁴-benzothiazin-4-yl)ethoxy]phenyl]-2-ethoxy propanoic acid phenyl glycinol salt;

(±)3-[4-[2-(3,4-Dihydro-2H-1,4-benzoxazin-4-yl)ethoxy]phenyl]-2-ethoxy propanoic acid phenyl glycinol salt;

(+)3-[4-[2-(3,4-Dihydro-2H-1,⁴-benzoxazin-4-yl)ethoxy]phenyl]-2-ethoxy propanoic acid phenyl glycinol salt;

(−)3-[4-[2-(3,4-Dihydro-2H-1,4-benzoxazin-4-yl)ethoxy]phenyl]-2-ethoxy propanoic acid phenyl glycinol salt;

(±)3-[4-[2-(3,4-Dihydro-2H-1,4-benzothiazin-4-yl)ethoxy]phenyl]-2-ethoxy propanoic acid amino guanidine salt;

(+)3-[4-[2-(3,4-Dihydro-2H-1,4-benzothiazin-4-yl)ethoxy]phenyl]-2-ethoxy propanoic acid amino guanidine salt;

(−)3-[4-[2-(3,4-Dihydro-2H-1,4-benzothiazin-4-yl)ethoxy]phenyl]-2-ethoxy propanoic acid amino guanidine salt;

(±)3-[4-[2-(3,4-Dihydro-2H-1,4-benzothiazin-4-yl)ethoxy]phenyl]-2-ethoxy propanoic acid amino guanidine salt;

(+)3-[4-[2-(3,4-Dihydro-2H-1,4-benzoxazin-4-yl)ethoxy]phenyl]-2-ethoxy propanoic acid amino guanidine salt;

(−)3-[4-[2-(3,4-Dihydro-2H-1,4-benzoxazin-4-yl)ethoxy]phenyl]-2-ethoxy propanoic acid amino guanidine salt;

(±)3-[4-[2-(3,4-Dihydro-2H-1,4-benzoxazin-4-yl)ethoxy]phenyl]-2-ethoxy propanoic acid methyl benzylamine salt;

(+)3-[4-[2-(3,4-Dihydro-2H-1,4-benzoxazin-4-yl)ethoxy]phenyl]-2-ethoxy propanoic acid methyl benzylamine salt;

(−)3-[4-[2-(3,4-Dihydro-2H-1,4-benzoxazin-4-yl)ethoxy]phenyl]-2-ethoxy propanoic acid methyl benzylamine salt;

(±)3-[4-[2-(3,4-Dihydro-2H-1,4-benzothiazin-4-yl)ethoxy]phenyl]-2-ethoxy propanoic acid methyl benzylamine salt;

(+)3-[4-[2-(3,4-Dihydro-2H-1,4-benzothiazin-4-yl)ethoxy]phenyl]-2-ethoxy propanoic acid methyl benzylamine salt;

(−)3-[4-[2-(3,4-Dihydro-2H-1,4-benzothiazin-4-yl)ethoxy]phenyl]-2-ethoxy propanoic acid methyl benzylamine salt;

(±)3-[4-[2-(3,4-Dihydro-2H-1,4-benzoxazin-4-yl)ethoxy]phenyl]-2-ethoxy propanoic acid amino guanidine hydrogen carbonate salt;

(+)3-[4-[2-(3,4-Dihydro-2H-1,4-benzoxazin-4-yl)ethoxy]phenyl]-2-ethoxy propanoic acid amino guanidine hydrogen carbonate salt;

(−)3-[4-[2-(3,4-Dihydro-2H-1,4-benzoxazin-4-yl)ethoxy]phenyl]-2-ethoxy propanoic acid amino guanidine hydrogen carbonate salt;

(±)3-[4-[2-(3,4-Dihydro-2H-1,4-benzothiazin-4-yl)ethoxy]phenyl]-2-ethoxy propanoic acid amino guanidine hydrogen carbonate salt;

(+)3-[4-[2-(3,4-Dihydro-2H-1,4-benzothiazin-4-yl)ethoxy]phenyl]-2-ethoxy propanoic acid amino guanidine hydrogen carbonate salt;

(−)3-[4-[2-(3,4-Dihydro-2H-1,4-benzothiazin-4-yl)ethoxy]phenyl]-2-ethoxy propanoic acid amino guanidine hydrogen carbonate salt;

(±)3-[4-[2-(3,4-Dihydro-2H-1,4-benzothiazin-4-yl)ethoxy]phenyl]-2-ethoxy propanoic acid tris(hydroxymethyl)aminomethane salt;

(+)3-[4-[2-(3,4-Dihydro-2H-1,4-benzothiazin-4-yl)ethoxy]phenyl]-2-ethoxy propanoic acid tris(hydroxymethyl)aminomethane salt;

(−)3-[4-[2-(3,4-Dihydro-2H-1,4-benzothiazin-4-yl)ethoxy]phenyl]-2-ethoxy propanoic acid tris(hydroxymethyl)aminomethane salt;

(±)3-[4-[2-(3,4-Dihydro-2H-1,4-benzoxazin-4-yl)ethoxy]phenyl]-2-ethoxy propanoic acid tris(hydroxymethyl)aminomethane salt;

(+)3-[4-[2-(3,4-Dihydro-2H-1,4-benzoxazin-4-yl)ethoxy]phenyl]-2-ethoxy propanoic acid tris(hydroxymethyl)aminomethane salt;

(−)3-[4-[2-(3,4-Dihydro-2H-1,4-benzoxazin-4-yl)ethoxy]phenyl]-2-ethoxy propanoic acid tris(hydroxymethyl)aminomethane salt;

(±)3-[4-[2-(3,4-Dihydro-2H-1,4-benzothiazin-4-yl)ethoxy]phenyl]-2-ethoxy propanoic acid N-octyl glucamine salt;

(+)3-[4-[2-(3,4-Dihydro-2H-1,4-benzothiazin-4-yl)ethoxy]phenyl]-2-ethoxy propanoic acid N-octyl glucamine salt;

(−)3-[4-[2-(3,4-Dihydro-2H-1,4-benzothiazin-4-yl)ethoxy]phenyl]-2-ethoxy propanoic acid N-octyl glucamine salt;

(±)3-[4-[2-(3,4-Dihydro-2H-1,4-benzoxazin-4-yl)ethoxy]phenyl]-2-ethoxy propanoic acid N-octyl glucamine salt;

(+)3-[4-[2-(3,4-Dihydro-2H-1,4-benzoxazin-4-yl)ethoxy]phenyl]-2-ethoxy propanoic acid N-octyl glucamine salt;

(−)3-[4-[2-(3,4-Dihydro-2H-1,4-benzoxazin-4-yl)ethoxy]phenyl]-2-ethoxy propanoic acid N-octyl glucamine salt;

(±)3-[4-[2-(3,4-Dihydro-2H-1,4-benzoxazin-4-yl)ethoxy]phenyl]-2-ethoxy propanoic acid N-methylglucamine salt;

(+)3-[4-[2-(3,4-Dihydro-2H-1,4-benzoxazin-4-yl)ethoxy]phenyl]-2-ethoxy propanoic acid N-methylglucamine salt;

(−)3-[4-[2-(3,4-Dihydro-2H-1,4-benzoxazin-4-yl)ethoxy]phenyl]-2-ethoxy propanoic acid N-methylglucamine salt;

(±)3-[4-[2-(3,4-Dihydro-2H-1,4-benzothiazin-4-yl)ethoxy]phenyl]-2-ethoxy propanoic acid N-methylglucamine salt;

(+)3-[4-[2-(3,4-Dihydro-2H-1,4-benzothiazin-4-yl)ethoxy]phenyl]-2-ethoxy propanoic acid N-methylglucamine salt; and

(−)3-[4-[2-(3,4-Dihydro-2H-1,4-benzothiazin-4-yl)ethoxy]phenyl]-2-ethoxy propanoic acid N-methylglucamine salt.

According, another feature of the present invention there is provided a process for the preparation of pharmaceutically acceptable salts of compound of the formula (I), which comprises, reacting the compound of the formula (III)

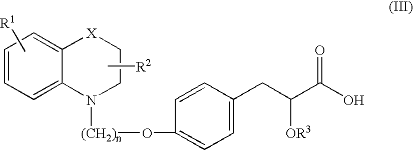

where all symbols are as defined earlier with a stoichiometric amount of an appropriate base in the presence of a solvent at a temperature in the range of −10° C. to the boiling point of the solvent employed for a period in the range of 10 minutes to 30 hours.

The compound of the formula (III) used may be either optically pure form or a racemic form. The base employed in the reaction may be selected from glucamine, N-methylglucamine, N-octylglucamine, dicyclohexylamine, methyl benzylamine, tris(hydroxymethyl)aminomethane (tromethamine), phenyl glycinol, lysine, aminoguanidine, aminoguanidine hydrogen carbonate or metformin. The solvent employed may be selected from alcohols such as ethanol, methanol, isopropanol, butanol and the like; ketones such as acetone, diethyl ketone, methyl ethyl ketone or their mixtures; ethers such as diethyl ether, ether, tetrahydrofuran, dioxane, dibutyl ether and the like or DMF, DMSO, xylene, toluene and the like or mixture thereof.

The pharmaceutically acceptable salts of the general formula (I) have significant formulation and bulk handling advantages in view of the their physicochemical properties and their stability.

Various polymorphs of a compound of general formula (I) forming part of this invention may be prepared by crystallization of compound of formula (I) under different conditions. For example, using different solvents commonly used or their mixtures for recrystallization; crystallizations at different temperatures; various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffraction or such other techniques.

The stereoisomers of the compounds forming part of this invention may be prepared by using compound of formula (I) in its single enantiomeric form in the process by resolving the mixture of stereoisomers by conventional methods. Some of the preferred methods include use of microbial resolution, resolving the diastereomeric salts formed with optically pure bases such as brucine, cinchona alkaloids and their derivatives, optically pure 2-alkyl phenethyl amine, phenyl glycinol and the like. The diastereomeric salts may be obtained in pure form by fractional crystallization. Commonly used methods are compiled by Jaques et al in “Enantiomers, Racemates and Resolution” (Wiley Isterscience, 1981).

Pharmaceutically acceptable solvates of the compounds of formula (I) forming part of this invention may be prepared by conventional methods such as dissolving the compounds of formula (I) in solvents such as water, methanol, ethanol and the like, preferably water and recrystallizing by using different crystallization techniques.

The present invention provides a pharmaceutical composition, containing the compounds of the general formula (I) as defined above, their derivatives, their analogs, their tautomeric forms, their stereoisomers, their polymorphs, their pharmaceutically acceptable solvates in combination with the usual pharmaceutically employed carriers, diluents and the like, useful for the treatment and/or prophylaxis of diseases such as hypertension, coronary heart disease, atherosclerosis, stroke, peripheral vascular diseases and related disorders. These compounds are useful for the treatment of familial hypercholesterolemia, hypertriglyceridemia, lowering of atherogenic lipoproteins, VLDL and LDL. The compounds of the present invention can be used for the treatment of certain renal diseases including glomerulonephritis, glomerulosclerosis, nephrotic syndrome, hypertensive nephrosclerosis, nephropathy. The compounds of general formula (I) are also useful for the treatment/prophylaxis of insulin resistance (type II diabetes), leptin resistance, impaired glucose tolerance, dyslipidemia, disorders related to syndrome X such as hypertension, obesity, insulin resistance, coronary heart disease, and other cardiovascular disorders. These compounds may also be useful as aldose reductase inhibitors, for improving cognitive functions in dementia, as inflammatory agents, treating diabetic complications, disorders related to endothelial cell activation, psoriasis, polycystic ovarian syndrome (PCOS), inflammatory bowel diseases, osteoporosis, myotonic dystrophy, pancreatitis, retinopathy, arteriosclerosis, xanthoma and for the treatment of cancer. The compounds of the present invention are useful in the treatment and/or prophylaxis of the above said diseases in combination/concomittant with one or more HMG CoA reductase inhibitors, hypolipidemic/ hypolipoproteinemic agents such as fibric acid derivatives, nicotinic acid, cholestyramine, colestipol, probucol or their combination. The compounds of the present invention in combination with HMG CoA reductase inhibitors, hypolipidemic/hypolipoproteinemic agents can be administered together or within such a period to act synergistically. The HMG CoA reductase inhibitors may be selected from those used for the treatment or prevention of hyperlipidemia such as lovastatin, provastatin, simvastatin, fluvastatin, atorvastatin, cerivastatin and their analogs thereof. Suitable fibric acid derivative may be gemfibrozil, clofibrate, fenofibrate, ciprofibrate, benzafibrate and their analogs thereof.

The present invention also provides a pharmaceutical composition, containing the compounds of the general formula (I) as defined above, their derivatives, their analogs, their tautomeric forms, their stereoisomers, their polymorphs, their pharmaceutically acceptable solvates and one or more HMG CoA reductase inhibitors, hypolipidemic/hypolipoproteinemic agents such as fibric acid derivatives, nicotinic acid, cholestyramine, colestipol, probucol in combination with the usual pharmaceutically employed carriers, diluents and the like.

The pharmaceutical composition may be in the forms normally employed, such as tablets, capsules, powders, syrups, solutions, suspensions and the like, may contain flavorants, sweeteners etc. in suitable solid or liquid carriers or diluents, or in suitable sterile media to form injectable solutions or suspensions. Such compositions typically contain from 1 to 20%, preferably 1 to 10% by weight of active compound, the remainder of the composition being pharmaceutically acceptable carriers, diluents or solvents.

Suitable pharmaceutically acceptable carriers include solid fillers or diluents and sterile aqueous or organic solutions. The active ingredient will be present in such pharmaceutical compositions in the amounts sufficient to provide the desired dosage in the range as described above. Thus, for oral administration, the active ingredient can be combined with a suitable solid or liquid carrier or diluent to form capsules, tablets, powders, syrups, solutions, suspensions and the like. The pharmaceutical compositions, may, if desired, contain additional components such as flavourants, sweeteners, excipients and the like. For parenteral administration, the active ingredient can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable acid addition salts or salts with base of the compounds. Aqueous solutions with the active ingredient dissolved in polyhydroxylated castor oil may also be used for injectable solutions. The injectable solutions prepared in this manner can then be administered intravenously, intraperitoneally, subcutaneously, or intramuscularly, with intramuscular administration being preferred in humans.

For nasal administration, the preparation may contain the active ingredient of the present invention dissolved or suspended in a liquid carrier, in particular an aqueous carrier, for aerosol application. The carrier may contain additives such as solubilizing agents, such as propylene glycol, surfactants, absorption enhancers such as lecithin (phosphatidylcholine) or cyclodextrin or preservatives such as parabenes.

Tablets, dragees or capsules having talc and/or a carbohydrate carried binder and the like are particularly suitable for any oral application. Preferably, carriers for tablets, dragees or capsules include lactose, corn starch and/or potato starch. A syrup or elixir can be used in cases where a sweetened vehicle can be employed.

A typical tablet production method is exemplified below:

Tablet Production Example:

| | | | |
|---|---|---|---|
| a) | 1) Active ingredient | 30 | g |
| | 2) Lactose | 95 | g |
| | 3) Corn starch | 30 | g |
| | 4) Carboxymethyl cellulose | 44 | g |
| | 5) Magnesium stearate | 1 | g |
| | | 200 | g |

for 1000 tablets

The ingredients 1 to 3 are uniformly blended with water and granulated after drying under reduced pressure. The ingredient 4 and 5 are mixed well with the granules and compressed by a tabletting machine to prepare 1000 tablets each containing 30 mg of active ingredient.

| | | | |
|---|---|---|---|
| b) | 1) Active ingredient | 30 | g |
| | 2) Calcium phosphate | 90 | g |
| | 3) Lactose | 40 | g |
| | 4) Corn starch | 35 | g |
| | 5) Polyvinyl pyrrolidone | 3.5 | g |
| | 6) Magnesium stearate | 1.5 | g |
| | | 200 | g |

for 1000 tablets

The ingredients 1-4 are uniformly moistened with an aqueous solution of 5 and granulated after drying under reduced pressure. Ingredient 6 is added and granules are compressed by a tabletting machine to prepare 1000 tablets containing 30 mg of ingredient 1.

The compound of the formula (I) as defined above are clinically administered to mammals, including man, via either oral, nasal, pulmonary, transdermal or parenteral, rectal, depot, subcutaneous, intravenous, intraurethral, intramuscular, intranasal, ophthalmic solution or an ointment. Administration by the oral route is preferred, being more convenient and avoiding the possible pain and irritation of injection. However, in circumstances where the patient cannot swallow the medication, or absorption following oral administration is impaired, as by disease or other abnormality, it is essential that the drug be administered parenterally. By either route, the dosage is in the range of about 0.01 to about 100 mg/kg body weight of the subject per day or preferably about 0.01 to about 30 mg/kg body weight per day administered singly or as a divided dose. However, the optimum dosage for the individual subject being treated will be determined by the person responsible for treatment, generally smaller doses being administered initially and thereafter increments made to determine the most suitable dosage.

The invention is explained in detail in the examples given below which are provided by way of illustration only and therefore should not be construed to limit the scope of the invention.

### EXAMPLE1

### S-Phenyl glycinol salt of (−)-3-[4-[2-(3,4-dihydro-2H-1,4-benzothiazin-4-yl)ethoxylphenyl]-2-ethoxy propanoic acid

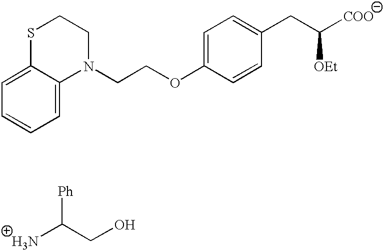

(−)-3-[4-[2-(3,4-Dihydro-2H-1,4-benzothiazin-4-yl)ethoxy]phenyl]-2-ethoxy propanoic acid (20.89 g) and isopropanol (210 ml) were added to 500 ml four necked round bottom flask, fitted with a mechanical stirrer and reflux condenser. The reaction mixture was heated slowly to 45-55° C. for complete dissolution of the glassy sticky mass. S-(+) phenyl glycinol (7.4 g) dissolved in isopropanol (75 ml) was added to the reaction mixture at 45-55° C. in about 30 min. under stirring. The reaction mixture was maintained for reflux at 80-90° C. for 12-14 h and monitored the progress of the reaction. The reaction mixture was brought to temperature of 45-50° C. under stirring and maintained for 2-3 hours at 45-55° C. The precipitated product was filtered, dried at 60° C. for 2-3 h to afford the pure S-phenyl glycinol salt of (−)-3-[4-[2-(3,4-Dihydro-2H-1,4-benzothiazin-4-yl)ethoxy]phenyl]-2-ethoxy propanoic acid as off-white to light cream color crystalline solid (weighs about 22 g, yield: 80%, m.p.: 126-128° C., purity 98-99% by HPLC).

IR (KBr) cm⁻¹: 3450-3300 (O—H stretch), 3060 (—C—H aromatic stretch), 2700-2200 (⁺NH₃ band), 2922 (—C—H aliphatic stretch), 1570 (—COO⁻ stretch), 1400 (—COO⁻ stretch).

¹H NMR (200 MHz, DMSO) δ: 1.0 (t, 3H, CH₃—CH₂—O); 2.6-3.40 (m, 5H, —S—CH₂, Ar—CH₂; CH—Ar), 3.45-4.0 (m, 8H, —CH₂—N—CH₂; CH₂—CH₂—O, CH₂—OH), 4.05 (q, 2H, —OCH₂), 4.3 (m, 1H, —CH—OEt), 6.5 (t, 1H, —CH₂—OH), 6.7-7.5 (m, 13H, Aromatic

Mass m/z: 388 (M⁺+1), 138 (C₈H₁₁O), 121 (C₈H₁₀).

Anal. Calcd for C₂₉H₃₆N₂O₅S, %C 66.41; %H 6.87; %N 5.34; Found %C 66.35, %H 6.74, %N 5.25.

### EXAMPLE 2

### Metformin salt of (−)-3-[4-[2-(3,4-dihydro-2H-1,4-benzothiazin-4-yl)ethoxy]phenyl]-2-ethoxy propanoic acid

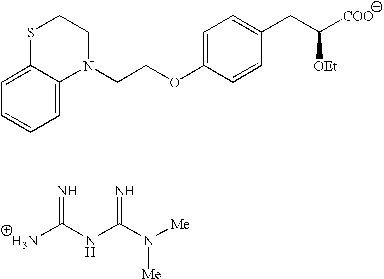

(−)-3-[4-[2-(3,4-Dihydro-2H-1,4-benzothiazin-4-yl)ethoxy]phenyl]-2-ethoxy propanoic acid (3.87 g) and isopropanol (40 ml) were added to 250 ml four necked round bottom flask, fitted with a mechanical stirrer and reflux condenser. The reaction mixture was slowly heated to 45-55° C. for complete dissolution of the glassy sticky mass. Metformin (1.29 g) dissolved in isopropanol (20 ml) was added to the reaction mixture at 55-65° C. in about 10 min. under stirring. The reaction mixture was maintained for reflux at 75-85° C. for 12-14 hours and monitored the progress of the reaction. The reaction mixture was cooled to room temperature and stirred for 2-3 h at room temperature. The precipitated product was filtered, dried at 60° C. for 2-3 h to afford the pure metformin salt of (−)-3-[4-2-(3,4-dihydro-2H-1,4-benzothiazin-4-yl)ethoxy]phenyl]-2-ethoxy propanoic acid as cream color crystalline solid (weighs about 3.1 g, yield: 78%, m.p.: 155-158° C., purity: 99% by HPLC).

IR (KBr) cm⁻¹: 3430-3300 (N—H stretch), 3053 (—C—H aromatic stretch), 2700-2200 (—NH₃ band), 2922 (—C—H aliphatic stretch), 1660 (—COO stretch), 1400 (—COO stretch).

¹H NMR (200 MHz, CD₃OD) δ: 1.0 (t, 3H, CH₃—CH₂—O), 2.6-3.40 (m, 11H, —S—CH₂, Ar—CH₂, CHAr, —NMe₂), 3.45-3.80 (m, 6H, —CH₂—N—CH₂,—CH₂—CH₂4.2 (t, 2H, —CH₂—CH₂—O), 6.5 (t, 1H, —CH₂—CH—), 6.65-7.2 (m, 8H, aromatic).

Mass m/z: 388 (M⁺+1), 130 (C₄H₁₁N₅), 113 (C₄H₈N₄).

Anal:Calcd.: C₂₅H₃₆N₆O₄S, %C 58.12; %H 6.97%, %N 16.3, Found %C 57.95%, %H 6.61, %N 16.25.

### EXAMPLE 3

### Dicyclohexylamine salt of (−)-3-[4-[2-(3,4-dihydro-2H-1,4-benzothiazin-4-yl)ethoxy]phenyl]-2-ethoxy propanoic acid

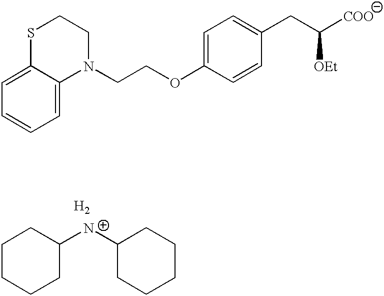

(−)-3-[4-[2-(3,4-Dihydro-2H-1,4-benzothiazin-4-yl)ethoxy]phenyl]-2-ethoxy propanoic acid (5.0 g) and isopropanol (50 ml) were added to 250 ml four necked round bottom flask fitted with a mechanical stirrer and reflux condenser. The reaction was slowly heated to 45-55° C. for complete dissolution of the glassy sticky mass. Dicyclohexylamine (2.33 g) in isopropanol (20 ml) was added to the reaction mixture at 55-65° C. in about 10 min. under stirring. The reaction mixture was maintained for reflux at 75-85° C. for 12-14 h and monitored the progress of the reaction by TLC. The reaction mixture was concentrated on rotavapor bath at 45-55° C. under reduced pressure to its half volume. The concentrated reaction mixture was cooled to RT and stirred for 2-3 h at room temperature. The precipitated product was filtered, dried at 60° C. for 2-3 h to afford the pure dicylcohexylamine salt of (−)-3-[4-[2-(3,4-dihydro-2H-1,4-benzothiazin-4-yl)ethoxy]phenyl]-2-ethoxy propanoic acid as off-white crystalline solid (weighs about 5.1 g, yield: 70%, m.p.: 110° C., purity by HPLC: 98-99%).

IR (KBr) cm⁻¹: 2932 (C—H aliphatic stretch), 2700-2200 (—NH₃ bands), 1582 (—COO stretch).

¹H NMR (200 MHz, DMSO-d₆) δ: 1.0 (t, 3H, CH₃—CH₂—O), 1.2-2.0 (m, 22H, Cyclohexyl), 2.4-3.4 (m, 5H, —S—CH₂, Ar—CH₂, —CH—Ar), 3.45-4.0 (m, 7H, —CH₂—N—CH₂—, CHOEt, CH₂—CH₂—O—), 4.05 (q, 2H, —OCH₂), 6.5 (t, 1H, —CH₂—CH—), 6.7-7.74 (m, 8H, aromatic).

Mass m/z: 388 (M⁺+1) 182 (C₁₂H₂₃N).

Anal: Calcd.: C₃₃H₄₈N₂O₄S, %C 69.71; %H 8.45%, %N 4.92, Found %C 69.60%, %H 8.35, %N 4.75.

### EXAMPLE 4

### (R)-(+)-Methyl benzylamine salt of (−)-3-[4-[2-(3,4-dihydro-2H-1,4-benzothiazin-4-yl)ethoxy]phenyl]-2-ethoxy propanoic acid

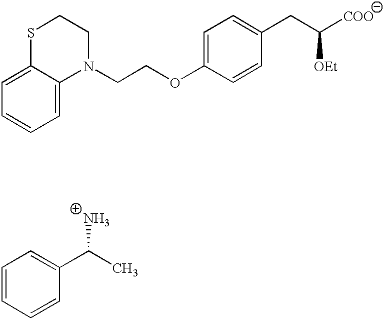

(−)-3-[4-[2-(3,4-Dihydro-2H-1,4-benzothiazin-4-yl)ethoxy]phenyl]-2-ethoxy propanoic acid (5.0 g) and isopropanol (50 ml) were added to 250 ml four necked round bottom flask fitted with a mechanical stirrer and reflux condenser. The reaction was slowly heated to 45-55° C. for complete dissolution of the glassy sticky mass. R-(+)-Methyl benzylamine (1.5 g) in isopropanol (20 ml) was added to the reaction mixture of 55-65° C. in about 10 min. under stirring. The reaction mixture was maintained for reflux at 75-85° C. for 12-14 h and monitored the progress of the reaction. The reaction mixture was cooled to 25-35° C. and stirred for 2-3 h. The precipitated product was filtered, dried at 60° C. for 2-3 h to afford the pure (R)-(+)-methylbenzylamine salt of (−)-3-[4-[2-(3,4-dihydro-2H-1,4-benzothiazin-4-yl)ethoxy]phenyl]-2-ethoxypropanoic acid as off-white crystalline solid (weighs about 6 g, yield: 91%, m.p. 126-128° C.; purity: 98.56-99.3% by HPLC).

IR (KBr) cm⁻¹: 2983-2856 (—N⁺H stretch), 1637(—COO, Stretch).

¹H NMR (200 MHz, CD₃OD) δ: 1.1 (t, 3H, CH₃—CH₂—O), 1.6 (d, 3H, CH₃—CH—), 2.6-3.4 (m, 5H, —S—CH₂—; Ar—CH₂, —CH—Ar), 3.45-4.0 (m, 7H, —CH₂N—CH₂—; —CH—OEt, CH₂—CH₂—O), 4.05 (q, 2H, —O—CH₂—) 6.5 (t, 1H, CH₂CH —CH₂—N—CH₂), 6.7-7.4 (m, 13H, aromatic).

Mass m/z: 388 (M⁺+1), 121(C₈H₁₁N), 105 (C₈H₈)

Anal: Calcd.: C₂₉H₃₆N₂O₄S, %C 68.50; %H 7.08%, %N 5.51, Found %C 68.38, %H 6.9, %N 5.4.

### EXAMPLE 5

### -Lysine salt of (−)-3-[4-[2-(3,4-dihydro-2H-1,4-benzothiazin-4-yl)ethoxy]phenyl]-2-ethoxy propanoic acid

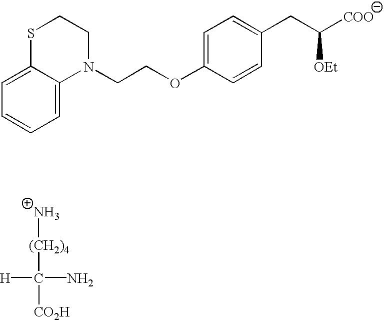

(−)-3-[4-[2-(3,4-Dihydro-2H-1,4-benzothiazin-4-yl)ethoxy]phenyl]-2-ethoxy propanoic acid (2.5 g) and isopropanol (25 ml) were added to the 100 ml four necked round bottom flask, fitted with a mechanical stirrer and reflux condenser. The reaction mixture was slowly heated to 45-55° C. for complete dissolution of the glassy sticky mass. L-Lysine monohydrate (1.0 g) dissolved in water (5 ml) was added to the reaction mixture at 45-55° C. in about 10 min. under stirring. The reaction mixture was maintained for reflux at 80-90° C. for 20-24 hrs and monitored the progress of the reaction. The isopropanol was distilled off along with azeotropic distillation of water using Dean-Stark apparatus. Fresh isopropanol (25 ml) was added to the residual reaction mixture and cooled the mixture initially to room temperature followed by cooling to 0-5° C. under stirring for 60-90 min. The precipitated product was filtered, dried at 60° C. for 2-3 hours to afford the pure L-lysine salt of (−)-3-[4-[2-(3,4-dihydro-2H-1,4-benzothiazin-4-yl)ethoxy]phenyl]-2-ethoxy propanoic acid as off white crystalline, hygroscopic solid (weighs about 2.5 g, yield: 78%, m.p. 142-144° C., purity 97.6-99.01% by HPLC).

IR (KBr) cm⁻¹: 3430-3400 (N—H stretch), 2920 (—C—H aliphatic stretch), 2700-2200 (—-N⁺H₃ stretch), 1585 (—COO⁻ stretch), 1400 (—COO⁻ stretch).

¹H NMR spectrum in DMSO-d₆+TFA (TMS as internal standard) is in confirmation with the assigned structure.

Mass m/z: 388 (M⁺+1), 164 (C₆H₁₆N₂O₃), 147 (C₆H₁₃NO₃).

Anal. Calcd for C₂₇H₄₁N₃O₇S; %C: 58.8; %H 7.44%; %N 7.62%, Found %C 58.7; %H 7.28; %N 7.55.

### EXAMPLE 6

### Amino guanidine hydrogen carbonate salt of (−)-3-[4-[2-(3,4-dihydro-2H-1,4-benzothiazin-4-yl)ethoxy]phenyl]-2-ethoxy propanoic acid

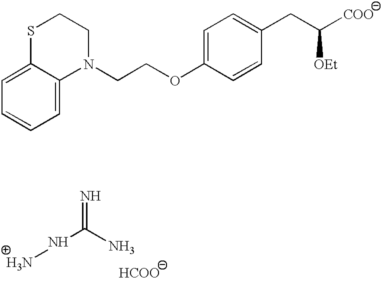

(−)-3-[4-[2-(3,4-Dihydro-2H-1,4-benzothiazin-4-yl)ethoxy]phenyl]-2-ethoxy propanoic acid (5.8 g) and methanol (60 ml) were added to 250 ml four necked round bottom flask, fitted with a mechanical stirrer and reflux condenser. The reaction mixture was slowly heated to 45-55° C. for complete dissolution of the glassy sticky mass. Amino guanidine hydrogen carbonate (2.0 g) dissolved in methanol (20 ml) was added to the reaction mixture at 45-55° C. in about 10 min. under stirring. The reaction mixture was maintained for reflux at 60-70° C. for 20-24 hours and monitored the progress of the reaction. The methanol was distilled off under reduced pressure at 40-50° C. and diisopropyl ether (50 ml) was added, filtered under nitrogen atmosphere. The red colored fluffy mass was further dried at 50-60° C. under high vacuum to afford very hygroscopic aminoguanidine salt of (−)-3-[4-[2-(3,4-dihydro-2H-1,4-benzothiazin-4-yl)ethoxy]phenyl]-2-ethoxy propanoic acid as red solid, (weighs about 6.0 g, yield: 80%, purity 97-99% by HPLC).

IR (KBr) cm⁻¹: 3400-3300 (N—H stretch), 2920 (—C—H aliphatic stretch), 1680 (—COO⁻ stretch), 1585 (—COO⁻ stretch), 1395 (—COO⁻ stretch).

¹H NMR (200 MHz, DMSO-d₆) δ: 1.0 (t, 3H, CH₃—CH₂—O), 2.6-3.4 (m, 5H, —S—CH₂—, Ar—CH₂, —CH—Ar), 3.45-4.0 (m, 7H, —CH₂—N—CH₂—, —CH—OEt, CH₂—CH₂—O—), 4.05 (q, 2H, —O—CH₂—), 6.5 (t, 1H, —CH₂—CH—), 6.7-7.4 (m, 8H, aromatic).

Mass m/z: 388 (M⁺+1), 136 (C₂H₈N₄O₂).

### EXAMPLE 7

### Tris(hydroxymethyl)aminomethane salt of (−)-3-[4-[2-(3,4-dihydro-2H-1,4-benzothiazin-4-yl)ethoxy]phenyl]-2-ethoxy propanoic acid

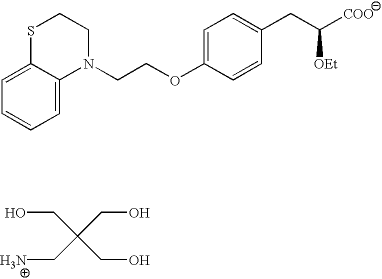

(−)-3-[4-[2-(3,4-Dihydro-2H-1,4-benzothiazin-4-yl)ethoxy]phenyl]-2-ethoxy propanoic acid (5.8 g) and methanol (60 ml) were added to 250 ml of four necked round bottom flask, fitted with a mechanical stirrer and reflux condenser. The reaction mixture was slowly heated to 45-55° C. for complete dissolution of the glassy sticky mass. Tris(hydroxymethyl)aminomethane (1.81 g) dissolved in methanol (10 ml) was added at 45-55° C. in about 10 min. under stirring. The reaction mixture was maintained for reflux at 60-80° C. for 20-24 h and monitored the progress of the reaction. The methanol was distilled off under reduced pressure at 40-50° C. diisopropyl ether (50 ml) was added and stirred for 10 min. The ether layer was decanted. The ether washing was repeated twice to afford the title compound as dark brown highly sticky mass (weighs about 7.0 g, yield: 90%, purity: 95-99% by HPLC).

IR (KBr) cm⁻¹: 3500-3300 (—N—H, O—H stretch), 2920 (—C—H stretch), 1585 (—COO⁻ stretch), 1409 (—COO⁻ stretch).

¹H NMR spectrum in DMSO-d₆+TFA (TMS as internal standard) is in conformity with the assigned structure.

Mass m/z: 388 (M⁺+1), 121 (C₄H₁₁NO₃).

### EXAMPLE-8

### N-Octyl glucamine salt of (−)-3-[4-[2-(3,4-dihydro-2H-1,4-benzothiazin-4-yl)ethoxy]phenyl]-2-ethoxy propanoic acid

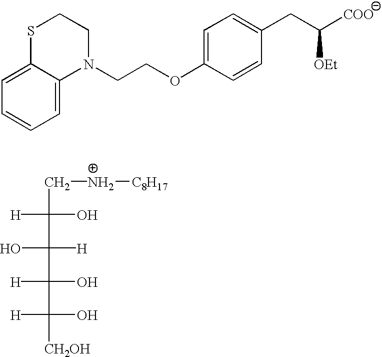

(−)-3-[4-[2-(3,4-Dihydro-2H-1,4-benzothiazin-4-yl)ethoxy]phenyl]-2-ethoxy propanoic acid (5.8 g) and methanol (60 ml) were added to 250 ml of four necked round bottom flask, fitted with a mechanical stirrer and reflux condenser. The reaction mixture was slowly heated to 45-55° C. for complete dissolution of the glassy sticky mass. N-Octyl glucamine (4.4 g) dissolved in methanol (25 ml) was added at 45-55° C. in about 10 min. under stirring. The reaction mixture was maintained for reflux at 60-70° C. for 20-24 h and monitored the progress of the reaction. The methanol was distilled off under reduced pressure at 40-50° C. and diisopropyl ether (50 ml) was added and stirred for 10 min. The ether layer was decanted and repeated the ether washing twice to afford the title compound as dark brown sticky mass, (weights about 8.0 g, yield 88%, purity: 96.5-99% by HPLC).

IR (KBr) cm⁻¹: 3350-3300 (—N—H stretch), 2920 (—C—H stretch), 1586 (—COO⁻ stretch), 1406 (—COO⁻ stretch).

¹H NMR spectrum is DMSO-d₆+TFA (TMS as internal standard) is in confirmation with the assigned structure.

Mass m/z: 388 (M⁺+1), 293 (C₁₄H₃₁NO₅).

### EXAMPLE-9

### N-methylglucamine salt of (−)-3-[4-[2-(3,4-dihydro-2H-1,4-benzothiazin-4-yl)ethoxy]phenyl]-2-ethoxy propanoic acid

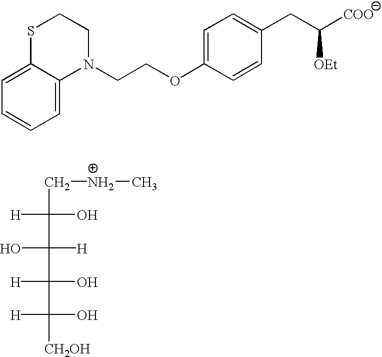

(−)-3-[4-[2-(3,4-Dihydro-2H-1,4-benzothiazin-4-yl)ethoxy]phenyl]-2-ethoxy propanoic acid (5.8 g) and methanol (60 ml) were added to 250 ml of four necked round bottom flask, fitted with a mechanical stirrer and reflux condenser. The reaction mixture was slowly heated to 45-55° C. for complete dissolution of the glassy sticky mass. N-methyl glucamine (2.92 g) dissolved in methanol (15ml) was added at 45-55° C. in about 10 min. under stirring. The reaction mixture was maintained for reflux at 60-70° C. for 20-24 h and monitored the progress of the reaction by TLC. The methanol was distilled off under reduced pressure at 40-50° C. and diisopropyl ether (50 ml) was added and stirred for 10 min. The ether layer was decanted and repeat the ether washing twice to afford the title compound as dark brown sticky mass, (weighs about 6.5 g, yield: 75%, purity 97.3-99% by HPLC). The purity of the salt depends on the purity of the acid used.

IR (KBr) cm⁻¹: 3350-3300 (—NH, —OH stretching), 2920 (C—H stretch), 1586 (—COO⁻ stretch).

¹H NMR spectrum in DMSO-d₆+TFA (TMS as internal standard) is in conformity with the assigned structure.

Mass m/z: 388 (M⁺+1), 195 (C₇H₁₁NO₅).

The compounds of the present invention lowered random blood sugar level, triglyceride, total cholesterol, LDL, VLDL and increased HDL. This was demonstrated by in vitro as well as in vivo animal experiments.

Demonstration of Efficacy of Compounds

A) In vitro:

a) Determination of hPPARα Activity

Ligand binding domain of hPPARα was fused to DNA binding domain of Yeast transcription factor GAL4 in eucaryotic expression vector. Using superfect (Qiagen, Germany) as transfecting reagent HEK-293 cells were transfected with this plasmid and a reporter plasmid harboring the luciferase gene driven by a GAL4 specific promoter. Compound was added at different concentrations after 42 hrs of transfection and incubated overnight. Luciferase activity as a function of compound binding/activation capacity of PPARα was measured using Packard Luclite kit (Packard, USA) in Top Count (Ivan Sadowski, Brendan Bell, Peter Broag and Melvyn Hollis. Gene. 1992. 118: 137 -141; Superfect Transfection Reagent Handbook. February 1997, Qiagen, Germany).

b) Determination of hPPARγ Activity

Ligand binding domain of hPPARγ1 was fused to DNA binding domain of Yeast transcription factor GAL4 in eucaryotic expression vector. Using lipofectamine (Gibco BRL, USA) as transfecting reagent HEK-293 cells were transfected with this plasmid and a reporter plasmid harboring the luciferase gene driven by a GAL4 specific promoter. Compound was added at 1 μM concentration after 48 hrs of transfection and incubated overnight. Luciferase activity as a function of drug binding/activation capacity of PPARγ1 was measured using Packard Luclite kit (Packard, USA) in Packard Top Count (Ivan Sadowski, Brendan Bell, Peter Broag and Melvyn Hollis. Gene. 1992. 118: 137-141; Guide to Eukaryotic Transfections with Cationic Lipid Reagents. Life Technologies, GIBCO BRL, USA).

| Example No. | Concentration | PPARα | PPARγ | Concentration |
|---|---|---|---|---|
| 2 | 50 μM | 3.0 | 1 μM | 9.8 |
| 3 | 50 μM | 5.2 | 1 μM | 19 |
| 5 | 50 μM | 3.5 | 1 μM | 7.6 |
| 6 | 50 μM | 4.7 | 1 μM | 21 |

c) Determination of HMG CoA Reductase Inhibition Activity

Liver microsome bound reductase was prepared from 2% cholestyramine fed rats at mid-dark cycle. Spectrophotometric assays were carried out in 100 mM KH₂PO₄, 4 mM DTT, 0.2 mM NADPH, 0.3 mM HMG CoA and 1251μg of liver microsomal enzyme. Total reaction mixture volume was kept as 1 ml. Reaction was started by addition of HMG CoA. Reaction mixture was incubated at 37° C. for 30 min and decrease in absorbance at 340 nm was recorded. Reaction mixture without substrate was used as blank (Goldstein, J. L and Brown, M. S. Progress in understanding the LDL receptor and HMG CoA reductase, two membrane proteins that regulate the plasma cholesterol. J. Lipid Res. 1984, 25: 1450-1461). The test compounds are expected to inhibit the HMG CoA reductase enzyme.

B) In vivo:

a) Efficacy in Genetic Models

Mutation in colonies of laboratory animals and different sensitivities to dietary regimens have made the development of animal models with non-insulin dependent diabetes and hyperlipidemia associated with obesity and insulin resistance possible. Genetic models such as db/db and ob/ob (Diabetes, (1982)31(1): 1-6) mice and zucker fa/fa rats have been developed by the various laboratories for understanding the pathophysiology of disease and testing the efficacy of new antidiabetic compounds (Diabetes, (1983)32: 830-838; Annu. Rep. Sankyo Res. Lab. (1994).46:1-57). The homozygous animals, C57 BL/KsJ-db/db mice developed by Jackson Laboratory, US, are obese, hyperglycemic, hyperinsulinemic and insulin resistant (J. Clin. Invest., (1990) 85: 962-967), whereas heterozygous are lean and normoglycemic. In db/db model, mouse progressively develops insulinopenia with age, a feature commonly observed in late stages of human type II diabetes when blood sugar levels are insufficiently controlled. The state of pancreas and its course vary according to the models. Since this model resembles that of type II diabetes mellitus, the compounds of the present invention were tested for blood sugar and triglycerides lowering activities.

Male C57BL/KsJ-db/db mice of 8 to 14 weeks age, having body weight range of 35 to 60 grams, bred at Dr. Reddy's Research Foundation (DRF) animal house, were used in the experiment. The mice were provided with standard feed (National Institute of Nutrition (NIN), Hyderabad, India) and acidified water, ad libitum. The animals having more than 350 mg/dl blood sugar were used for testing. The number of animals in each group was 4.

Test compounds were suspended on 0.25% carboxymethyl cellulose and administered to test group at a dose of 0.1 mg to 30 mg/kg through oral gavage daily for 6 days. The control group received vehicle (dose 10 ml/kg). On 6th day the blood samples were collected one hour after administration of test compounds/vehicle for assessing the biological activity.

The random blood sugar and triglyceride levels were measured by collecting blood (100 μl) through orbital sinus, using heparinised capillary in tubes containing EDTA which was centrifuged to obtain plasma. The plasma glucose and triglyceride levels were measured spectrometrically, by glucose oxidase and glycerol 3 PO₄ oxidase/peroxidase enzyme (Dr. Reddy's Lab. Diagnostic Division Kits, Hyderabad, India) methods respectively.

The blood sugar and triglycerides lowering activities of the test compound was calculated according to the formula.

No adverse effects were observed for any of the mentioned compounds of invention in the above test.

| | | Reduction in Blood | Triglyceride |
|---|---|---|---|
| Compound | Dose (mg/kg) | Glucose Level (%) | Lowering (%) |
| Example 2 | 0.03 | 56 | 59 |

The ob/ob mice were obtained at 5 weeks of age from Bomholtgard, Denmark and were used at 8 weeks of age. Zucker fa/fa fatty rats were obtained from IffaCredo, France at 10 weeks of age and were used at 13 weeks of age. The animals were maintained under 12 hour light and dark cycle at 25+1° C. Animals were given standard laboratory chow (NIN, Hyderabad, India) and water, ad libitum (Fujiwara, T., Yoshioka, S., Yoshioka, T., Ushiyama, I and Horikoshi, H. Characterization of new oral antidiabetic agent CS-045, Studies in KK and ob/ob mice and Zucker fatty rats. Diabetes. 1988. 37:1549-1558).

The test compounds were administered at 0.1 to 30 mg/kg/day dose for 9 days.

The control animals received the vehicle (0.25% carboxymethylcellulose, dose 10 ml/kg) through oral gavage.

The blood samples were collected in fed state 1 hour after drug administration on 0 and 9 day of treatment. The blood was collected from the retro-orbital sinus through heparinised capillary in EDTA containing tubes. After centrifugation, plasma sample was separated for triglyceride, glucose, free fatty acid, total cholesterol and insulin estimations. Measurement of plasma triglyceride, glucose, total cholesterol were done using commercial kits (Dr. Reddy's Laboratory, Diagnostic Division, India). The plasma free fatty acid was measured using a commercial kit from Boehringer Mannheim, Germany. The plasma insulin was measured using a RIA kit (BARC, India). The reduction of various parameters examined are calculated according to the formula given below.

In ob/ob mice oral glucose tolerance test was performed after 9 days treatment. Mice were fasted for 5 hrs and challenged with 3 gm/kg of glucose orally. The blood samples were collected at 0, 15, 30, 60 and 120 min for estimation of plasma glucose levels.

The experimental results from the db/db mice, ob/ob mice, Zucker fa/fa rats suggest that the novel compounds of the present invention also possess therapeutic utility as a prophylactic or regular treatment for diabetes, obesity, cardiovascular disorders such as hypertension, hyperlipidaemia and other diseases; as it is known from the literature that such diseases are interrelated to each other.

Blood glucose level and triglycerides are also lowered at doses greater than 10 mg/kg. Normally, the quantum of reduction is dose dependent and plateaus at certain dose.

b) Plasma Triglyceride and Cholesterol Lowering Activity in Hypercholesterolemic Rat Models

Male Sprague Dawley rats (NIN stock) were bred in DRF animal house. Animals were maintained under 12 hour light and dark cycle at 25±1° C. Rats of 180-200 gram body weight range were used for the experiment. Animals were made hypercholesterolemic by feeding 2% cholesterol and I% sodium cholate mixed with standard laboratory chow [National Institute of Nutrition (NIN), Hyderabad, India] for 6 days. Throughout the experimental period the animals were maintained on the same diet (Petit, D., Bonnefis, M. T., Rey, C and Infante, R. Effects of ciprofibrate on liver lipids and lipoprotein synthesis in normal and hyperlipidemic rats. Atherosclerosis. 1988. 74: 215-225).

The test compounds were administered orally at a dose 0.1 to 30 mg/kg/day for 3 days. Control group was treated with vehicle alone (0.25% Carboxymethylcellulose; dose 10 ml/kg).

The blood samples were collected in fed state 1 hour after drug administration on 0 and 3 day of compound treatment. The blood was collected from the retro-orbital sinus through heparinised capillary in EDTA containing tubes. After centrifugation, plasma sample was separated for total cholesterol, HDL and triglyceride estimations. Measurement of plasma triglyceride, total cholesterol and HDL were done using commercial kits (Dr. Reddy's Laboratory, Diagnostic Division, India). LDL and VLDL cholesterol were calculated from the data obtained for total cholesterol, HDL and triglyceride. The reduction of various parameters examined are calculated according to the formula.

c) Plasma Triglyceride and Total Cholesterol Lowering Activity in Swiss Albino Mice and Guinea Pigs

Male Swiss albino mice (SAM) and male Guinea pigs were obtained from NIN and housed in DRF animal house. All these animals were maintained under 12 hour light and dark cycle at 25±1° C. Animals were given standard laboratory chow (NIN, Hyderabad, India) and water, ad libitum. SAM of 20-25 g body weight range and Guinea pigs of 500-700 g body weight range were used (Oliver, P., Plancke, M. O., Marzin, D., Clavey, V., Sauzieres, J and Fruchart, J. C. Effects of fenofibrate, gemfibrozil and nicotinic acid on plasma lipoprotein levels in normal and hyperlipidemic mice. Atherosclerosis. 1988. 70: 107-114).

The test compounds were administered orally to Swiss albino mice at 0.3 to 30 mg/kg/day dose for 6 days. Control mice were treated with vehicle (0.25% Carboxymethylcellulose; dose 10 ml/kg). The test compounds were administered orally to Guinea pigs at 0.3 to 30 mg/kg/day dose for 6 days. Control animals were treated with vehicle (0.25% Carboxymethylcellulose; dose 5 ml/kg).

The blood samples were collected in fed state 1 hour after drug administration on 0 and 6 day of treatment. The blood was collected from the retro-orbital sinus through heparinised capillary in EDTA containing tubes. After centrifugation, plasma sample was separated for triglyceride and total cholesterol (Wieland, O. Methods of Enzymatic analysis. Bergermeyer, H. O., Ed., 1963. 211-214; Trinder, P. Ann. Clin. Biochem. 1969. 6: 24-27). Measurement of plasma triglyceride, total cholesterol and HDL were done using commercial kits (Dr. Reddy's Diagnostic Division, Hyderabad, India).

| Compound | Dose (mg/kg) | Triglyceride Lowering (%) |
|---|---|---|
| 1 | 3 | 66 |
| 2 | 3 | 55 |
| 4 | 3 | 55 |
| 5 | 3 | 46 |

c) Body Weight Reducing Effect in Cholesterol Fed Hamsters

Male Syrian Hamsters were procured from NIN, Hyderabad, India. Animals were housed at DRF animal house under 12 hour light and dark cycle at 25±1° C. with free access to food and water. Animals were maintained with 1% cholesterol containing standard laboratory chow (NIN) from the day of treatment.

The test compounds were administered orally at 1 to 30 mg/kg/day dose for 15 days. Control group animals were treated with vehicle (Mill Q water, dose 10 ml/kg/day). Body weights were measured on every 3^{rd} day.

Formulae for Calculation

1. Percent reduction in Blood sugar/triglycerides/total cholesterol were calculated according to the formula:

$$Percent reduction ( \% ) = [ 1 - \frac{TT / OT}{TC / OC} ] \times 100$$

OC=Zero day control group value

OT=Zero day treated group value

TC=Test day control group value

TT=Test day treated group value

2. LDL and VLDL cholesterol levels were calculated according to the formula:

$$LDL cholesterol in mg \text{/} dl = \\ [ Total cholesterol - HDL cholesterol - \frac{Triglyceride}{5} ] mg \text{/} dl$$

VLDL cholesterol in mg/dl=[Total cholesterol−HDL cholesterol−LDL cholesterol] mg/dl.

Single Dose Oral Pharmacokinetic Studies

Male Wistar rats (220-250 gm) were used in the experiments. The animals were maintained under standard laboratory conditions and had free access to feed and water ad libitum. Before experimentation animals were fasted overnight (˜15 h) during which they had free access to water ad libitum.

An amount equivalent to 30 mg of drug was weighed accurately and transferred into a clean mortar and triturated to obtain a fine powder. To this 0.5 ml of 0.25% sodium carboxy methyl cellulose (sodium CMC) was added to obtain a paste. To the obtained paste remaining 2.5 ml of sodium CMC was added to make up the volume to 3 ml. Based on the animal weight appropriate volume (body weight×3) of the prepared suspension was administered through oral gavage.

After dosing, at designated time points (0.5, 1, 2, 3, 5, 8, 12 and 24 h) 200 μl of blood was collected from retro orbital plexus into 0.5 ml eppendorff tubes containing EDTA (10 μl of 200 mg/ml solution in Milli Q water). Blood was centrifuged at 12,800 rpm for 5 min and obtained plasma and stored at −20° C. till further analysis.

100 μl plasma was transferred into a clean and dry centrifuge tube. To this internal standard (10 μl of 100 μg/ml) was added and extracted with 2 ml of extraction recovery solvent. The contents were vortexed for 2 min, followed by centrifugation for 10 min at 2800 rpm. Clear organic layer (2×0.75 ml) was separated and dried under nitrogen gas at 50° C. The residue was reconstituted with 150 μl of mobile phase and vortexed for 20 sec, from this 50 μl was injected onto HPLC column.

Pharmacokinetic parameters were calculated by non-compartmental model analysis. The peak plasma concentration (Cₘₐₓ) and the corresponding time (Tₘₐₓ) were directly obtained from the raw data. The area under the plasma concentration versus time curve up to the last quantifiable time point, AUC₍₀₋ₜ₎ was obtained by the linear and log-linear trapezoidal summation. The AUC₍₀₋ₜ₎ extrapolated to infinity (i.e., AUC_{(0-∞)}) by adding the quotient of Cₗₐₛₜ/Kₑₗ, where Cₗₐₛₜ represents the last measurable time concentration and Kₑₗ represents the apparent terminal rate constant. Kₑₗ was calculated by the linear regression of the log-transformed concentrations of the drug in the terminal phase. The half-life of the terminal elimination phase was obtained using the relationship

t_{½}0.693/Kₑₗ.

| Example | AUC_{(0-∞)} | AUC₍₀₋ₜ₎ | Cₘₐₓ | | | |
|---|---|---|---|---|---|---|
| No. | (μg · hr/ml) | (μg · hr/ml) | (μg · hr/ml) | Tₘₐₓ (h) | Kₑ₁ (h⁻¹) | T_{1/2} (h) |
| 2 | 319.93 ± 36.19 | 315.05 ± 34.73 | 77.23 ± 24.07 | 0.63 ± 0.25 | 0.17 ± 0.03 | 4.25 ± 0.86 |

1. Pharmaceutically acceptable salts of compound of the general formula (I) 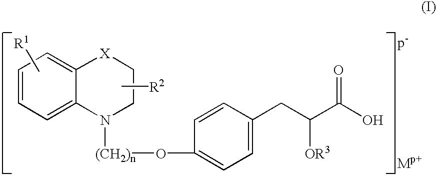
their derivatives, their analogs, their tautomeric forms, their stereoisomers, their polymorphs, wherein R¹ represents hydrogen, halogen, hydroxy, nitro, cyano or lower alkyl group; R²represents hydrogen, lower alkyl or oxo group; X represents a heteroatom selected from oxygen or sulfur; R³ represents hydrogen or lower alkyl group; n is an integer ranging from 1-4; M represents a counter ion or a moiety which forms a pharmaceutically acceptable salt; p is an integer ranging from 1 to 2.

2. A compound as claimed in claim 1, where in the groups represented by M is selected from glucamine, N-methylglucamine, N-octylglucamine, dicyclohexylamine, methyl benzylamine, tris(hydroxymethyl)aminomethane, phenyl glycinol, lysine, aminoguanidine, aminoguanidine hydrogen carbonate or metformin.

3. A process for the preparation of pharmaceutically acceptable salts of compound of the general formula (I) 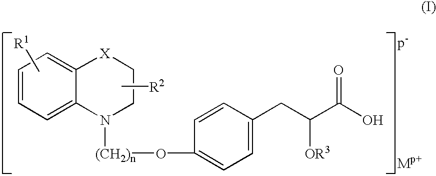
wherein R¹ represents hydrogen, halogen, hydroxy, nitro, cyano or lower alkyl group; R² represents hydrogen, lower alkyl or oxo group; X represents a heteroatom selected from oxygen or sulfur; R³ represents hydrogen or lower alkyl group; the linking group represented by —(CH₂)ₙ—O— may be attached either through a nitrogen atom or a carbon atom; n is an integer ranging from 1-4; M represents a counter ion or a moiety which forms a pharmaceutically acceptable salt; p is an integer ranging from 1 to 2, which comprises, reacting the compound of the formula (III) 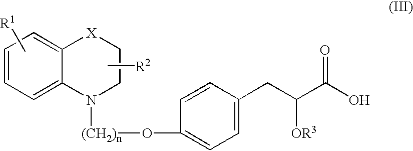
where all symbols are as defined above with a stoichiometric amount of a base in the presence of a solvent.

4. The process as claimed in claim 3, wherein the base used is selected from glucamine, N-methylglucamine, N-octylglucamine, dicyclohexylamine, methyl benzylamine, tris(hydroxymethyl)aminomethane, phenyl glycinol, lysine, aminoguanidine, aminoguanidine hydrogen carbonate or metformin.

5. The process as claimed in claims 3 and 4, wherein the solvent used is selected from an alcohol, ketone, ether, DMF, DMSO, xylene, toluene or a mixture thereof.

6. The process as claimed in claims 3 to 5, wherein the temperature of the reaction ranges from −10° C. to the boiling point of the solvent employed for a period in the range of 10 minutes to 30 hours.

7. A compound according to claim 1, which is selected from:
(±) 3-[4-[2-(3,4-Dihydro-2H-1,4-benzoxazin-4-yl)ethoxy]phenyl]-2-ethoxy propanoic acid lysine salt;
(+) 3-[4-[2-(3,4-Dihydro-2H-1,4-benzoxazin-4-yl)ethoxy]phenyl]-2-ethoxy propanoic acid lysine salt;
(−) 3-[4-[2-(3,4-Dihydro-2H-1,4-benzoxazin-4-yl)ethoxy]phenyl]-2-ethoxy propanoic acid lysine salt;
(±) 3-[4-[2-(3,4-Dihydro-2H-1,4-benzothiazin-4-yl)ethoxy]phenyl]-2-ethoxy propanoic acid lysine salt;
(+) 3-[4-[2-(3,4-Dihydro-2H-1,4-benzothiazin-4-yl)ethoxy]phenyl]-2-ethoxy propanoic acid lysine salt;
(−) 3-[4-[2-(3,4-Dihydro-2H-1,4-benzothiazin-4-yl)ethoxy]phenyl]-2-ethoxy propanoic acid lysine salt;
(±) 3-[4-[2-(3,4-Dihydro-2H-1,⁴-benzoxazin-4-yl)ethoxy]phenyl]-2-ethoxy propanoic acid dicyclohexylamine salt;
(+) 3-[4-[2-(3,4-Dihydro-2H-1,4-benzoxazin-4-yl)ethoxy]phenyl]-2-ethoxy propanoic acid dicyclohexylamine salt;
(−) 3-[4-[2-(3,4-Dihydro-2H-1,4-benzoxazin-4-yl)ethoxy]phenyl]-2-ethoxy propanoic acid dicyclohexylamine salt;
(±) 3-[4-[2-(3,4-Dihydro-2H-1,4-benzothiazin-4-yl)ethoxy]phenyl]-2-ethoxy propanoic acid dicyclohexylamine salt;
(+) 3-[4-[2-(3,4-Dihydro-2H-1,4-benzothiazin-4-yl)ethoxy]phenyl]-2-ethoxy propanoic acid dicyclohexylamine salt;
(−) 3-[4-[2-(3,4-Dihydro-2H-1,4-benzothiazin-4-yl)ethoxy]phenyl]-2-ethoxy propanoic acid dicyclohexylamine salt;
(±) 3-[4-[2-(3,4-Dihydro-2H-1,4-benzoxazin-4-yl)ethoxy]phenyl]-2-ethoxy propanoic acid metformin salt;
(+) 3-[4-[2-(3,4-Dihydro-2H-1,4-benzoxazin-4-yl)ethoxy]phenyl]-2-ethoxy propanoic acid metformin salt;
(−) 3-[4-[2-(3,4-Dihydro-2H-1,4-benzoxazin-4-yl)ethoxy]phenyl]-2-ethoxy propanoic acid metformin salt;
(±) 3-[4-[2-(3,4-Dihydro-2H-1,4-benzothiazin-4-yl)ethoxy]phenyl]-2-ethoxy propanoic acid metformin salt;
(+) 3-[4-[2-(3,4-Dihydro-2-1,4-benzothiazin-4-yl)ethoxy]phenyl]-2-ethoxy propanoic acid metformin salt;
(−) 3-[4-[2-(3,4-Dihydro-2H-1,4-benzothiazin-4-yl)ethoxy]phenyl]-2-ethoxy propanoic acid metformin salt;
(±) 3-[4-[2-(3,4-Dihydro-2H-1,4-benzothiazin-4-yl)ethoxy]phenyl]-2-ethoxy propanoic acid phenyl glycinol salt;
(+) 3-[4-[2-(3,4-Dihydro-2H-1,4-benzothiazin-4-yl)ethoxy]phenyl]-2-ethoxy propanoic acid phenyl glycinol salt;
(−) 3-[4-[2-(3,4-Dihydro-2H-1,4-benzothiazin-4-yl)ethoxy]phenyl]-2-ethoxy propanoic acid phenyl glycinol salt;
(±) 3-[4-[2-(3,4-Dihydro-2H-1,4-benzoxazin-4-yl)ethoxy]phenyl]-2-ethoxy propanoic acid phenyl glycinol salt;
(+) 3-[4-[2-(3,4-Dihydro-2H-1,4-benzoxazin-4-yl)ethoxy]phenyl]-2-ethoxy propanoic acid phenyl glycinol salt;
(−) 3-[4-[2-(3,4-Dihydro-2H-1,4-benzoxazin-4-yl)ethoxy]phenyl]-2-ethoxy propanoic acid phenyl glycinol salt;
(±) 3-[4-[2-(3,4-Dihydro-2H-1,4-benzothiazin-4-yl)ethoxy]phenyl]-2-ethoxy propanoic acid amino guanidine salt;
(+) 3-[4-[2-(3,4-Dihydro-2H-1,4-benzothiazin-4-yl)ethoxy]phenyl]-2-ethoxy propanoic acid amino guanidine salt;
(−) 3-[4-[2-(3,4-Dihydro-2H-1,4-benzothiazin-4-yl)ethoxy]phenyl]-2-ethoxy propanoic acid amino guanidine salt;
(±) 3-[4-[2-(3,4-Dihydro-2H-1,4-benzoxazin-4-yl)ethoxy]phenyl]-2-ethoxy propanoic acid amino guanidine salt;
(+) 3-[4-[2-(3,4-Dihydro-2H-1,4-benzoxazin-4-yl)ethoxy]phenyl]-2-ethoxy propanoic acid amino guanidine salt;
(−) 3-[4-[2-(3,4-Dihydro-2H-1,4-benzoxazin-4-yl)ethoxy]phenyl]-2-ethoxy propanoic acid amino guanidine salt;
(±) 3-[4-[2-(3,4-Dihydro-2H-1,4-benzoxazin-4-yl)ethoxy]phenyl]-2-ethoxy propanoic acid amino guanidine salt;
(+) 3-[4-[2-(3,4-Dihydro-2H-1,4-benzoxazin-4-yl)ethoxy]phenyl]-2-ethoxy propanoic acid methyl benzylamine salt;
(−) 3-[4-[2-(3,4-Dihydro-2H-1,4-benzoxazin-4-yl)ethoxy]phenyl]-2-ethoxy propanoic acid methyl benzylamine salt;
(±) 3-[4-[2-(3,4-Dihydro-2H-1,4-benzothiazin-4-yl)ethoxy]phenyl]-2-ethoxy propanoic acid methyl benzylamine salt;
(+) 3-[4-[2-(3,4-Dihydro-2H-1,4-benzothiazin-4-yl)ethoxy]phenyl]-2-ethoxy propanoic acid methyl benzylamine salt;
(−) 3-[4-[2-(3,4-Dihydro-2H-1,4-benzothiazin-4-yl)ethoxy]phenyl]-2-ethoxy propanoic acid methyl benzylamine salt;
(±) 3-[4-[2-(3,4-Dihydro-2H-1,4-benzoxazin-4-yl)ethoxy]phenyl]-2-ethoxy propanoic acid amino guanidine hydrogen carbonate salt;
(+) 3-[4-[2-(3,4-Dihydro-2H-1,4-benzoxazin-4-yl)ethoxy]phenyl]-2-ethoxy propanoic acid amino guanidine hydrogen carbonate salt;
(−) 3-[4-[2-(3,4-Dihydro-2H-1,4-benzoxazin-4-yl)ethoxy]phenyl]-2-ethoxy propanoic acid amino guanidine hydrogen carbonate salt;
(±) 3-[4-[2-(3,4-Dihydro-2H-1,4-benzothiazin-4-yl)ethoxy]phenyl]-2-ethoxy propanoic acid amino guanidine hydrogen carbonate salt;
(+) 3-[4-[2-(3,4-Dihydro-2H-1,4-benzothiazin-4-yl)ethoxy]phenyl]-2-ethoxy propanoic acid amino guanidine hydrogen carbonate salt;
(−) 3-[4-[2-(3,4-Dihydro-2H-1,4-benzothiazin-4-yl)ethoxy]phenyl]-2-ethoxy propanoic acid amino guanidine hydrogen carbonate salt;
(±) 3-[4-[2-(3,4-Dihydro-2H-1,4-benzothiazin-4-yl)ethoxy]phenyl]-2-ethoxy propanoic acid tris(hydroxymethyl)aminomethane salt;
(+) 3-[4-[2-(3,4-Dihydro-2H-1,4-benzothiazin-4-yl)ethoxy]phenyl]-2-ethoxy propanoic acid tris(hydroxymethyl)aminomethane salt;
(−) 3-[4-[2-(3,4-Dihydro-2H-1,4-benzothiazin-4-yl)ethoxy]phenyl]-2-ethoxy propanoic acid tris(hydroxymethyl)aminomethane salt;
(±) 3-[4-[2-(3,4-Dihydro-2H-1,4-benzoxazin-4-yl)ethoxy]phenyl]-2-ethoxy propanoic acid tris(hydroxymethyl)aminomethane salt;
(+) 3-[4-[2-(3,4-Dihydro-2H-1,4-benzoxazin-4-yl)ethoxy]phenyl]-2-ethoxy propanoic acid tris(hydroxymethyl)aminomethane salt;
(−) 3-[4-[2-(3,4-Dihydro-2H-1,4-benzoxazin-4-yl)ethoxy]phenyl]-2-ethoxy propanoic acid tris(hydroxymethyl)aminomethane salt;
(±) 3-[4-[2-(3,4-Dihydro-2H-1,4-benzoxazin-4-yl)ethoxy]phenyl]-2-ethoxy propanoic acid N-octyl glucamine salt;
(+) 3-[4-[2-(3,4-Dihydro-2H-1,4-benzoxazin-4-yl)ethoxy]phenyl]-2-ethoxy propanoic acid N-octyl glucamine salt;
(−) 3-[4-[2-(3,4-Dihydro-2H-1,4-benzoxazin-4-yl)ethoxy]phenyl]-2-ethoxy propanoic acid N-octyl glucamine salt;
(±) 3-[4-[2-(3,4-Dihydro-2H-1,4-benzoxazin-4-yl)ethoxy]phenyl]-2-ethoxy propanoic acid N-octyl glucamine salt;
(+) 3-[4-[2-(3,4-Dihydro-2H-1,4-benzoxazin-4-yl)ethoxy]phenyl]-2-ethoxy propanoic acid N-octyl glucamine salt;
(−) 3-[4-[2-(3,4-Dihydro-2H-1,4-benzoxazin-4-yl)ethoxy]phenyl]-2-ethoxy propanoic acid N-octyl glucamine salt;
(±) 3-[4-[2-(3,4-Dihydro-2H-1,4-benzoxazin-4-yl)ethoxy]phenyl]-2-ethoxy propanoic acid N-methylglucamine salt;
(+) 3-[4-[2-(3,4-Dihydro-2H-1,4-benzoxazin-4-yl)ethoxy]phenyl]-2-ethoxy propanoic acid N-methylglucamine salt;
(−) 3-[4-[2-(3,4-Dihydro-2H-1,4-benzoxazin-4-yl)ethoxy]phenyl]-2-ethoxy propanoic acid N-methylglucamine salt;
(±) 3-[4-[2-(3,4-Dihydro-2H-1,4-benzothiazin-4-yl)ethoxy]phenyl]-2-ethoxy propanoic acid N-methylglucamine salt;
(+) 3-[4-[2-(3,4-Dihydro-2H-1,4-benzothiazin-4-yl)ethoxy]phenyl]-2-ethoxy propanoic acid N-methylglucamine salt; and
(−) 3-[4-[2-(3,4-Dihydro-2H-1,4-benzothiazin-4-yl)ethoxy]phenyl]-2-ethoxy propanoic acid N-methylglucamine salt.

8. A composition, which comprises a compound of formula (I) 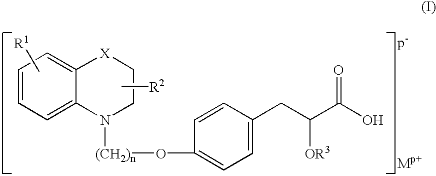
as defined in claim 1 or a compound as claimed in claim 7 and a pharmaceutically acceptable carrier, diluent, excipient or solvate.

9. A composition which comprises a compound of formula (I) as defined in claim 1 or a compound as claimed in claim 7 and an HMG CoA reductase inhibitor, fibrate, nicotinic acid, cholestyramine, cholestipol, probucol or a mixture thereof and a pharmaceutically acceptable carrier, diluent, excipient or solvate.

10. A composition as claimed in claims 8 and 9 in the form of a tablet, capsule, powder, syrup, solution or suspension.

11. A pharmaceutical composition as claimed in claims 8 and 9 for the treatment and/or prevention of type II diabetes, glucose intolerance, leptin resistance, dyslipidaemia, disorders related to Syndrome X such as hypertension, obesity, insulin resistance, atherosclerosis, hyperlipidemia, coronary artery disease and other cardiovascular disorders, certain renal diseases including glomerulonephritis, glomerulosclerosis, nephrotic syndrome, hypertensive nephrosclerosis, nephropathy, disorders related to endothelial cell activation, psoriasis, polycystic ovarian syndrome (PCOS), osteoporosis, inflammatory bowel diseases, myotonic dystrophy, pancreatitis, retinopathy, arteriosclerosis, xanthoma or cancer.

12. A method of preventing or treating hyperlipemia, hypercholesteremia, hyperglycemia, osteoporosis, obesity, impaired glucose tolerance, atherosclerosis, leptin resistance, insulin resistance or diseases in which insulin resistance is the underlying pathophysiological mechanism comprising administering a compound of formula (I) as defined in claim 1 or a compound as claimed in claim 7 or a pharmaceutical composition according to claim 8 or 9 to a patient in need thereof.

13. A method according to claim 12, wherein the disease is type II diabetes, impaired glucose tolerance, dyslipidemia, disorders related to Syndrome X including hypertension, obesity, insulin resistance, coronary artery disease and other cardiovascular disorders; renal diseases including glomerulonephritis, glomeruloscierosis, nephrotic syndrome, hypertensive nephrosclerosis, retinopathy, nephropathy, disorders to related endothelial cell activation, psoriasis, polycystic ovarian syndrome (PCOS), dementia, diabetic complications, inflammatory bowel diseases, myotonic dystrophy, pancreatitis, arteriosclerosis, xanthoma, eating disorders, cancer or osteoporosis or as inflammatory agents.

14. A method according to claim 12, for the treatment and/or prophylaxis of disorders related to Syndrome X, which comprises administering an agonist of PPARα and/or PPARγ of formula (I) as claimed in claim 1 or a compound as claimed in claim 7 or a pharmaceutical composition according to claim 8 or 9 to a patient in need thereof.

15. A method of reducing total cholesterol, body weight, blood plasma glucose, triglycerides, LDL, VLDL or free fatty acids or increasing HDL in the plasma comprising administering a compound of formula (I), as defined in claim 1 or a compound as claimed in claim 7 or a pharmaceutical composition according to claim 8 or 9 to a patient in need thereof.

16. A method of preventing or treating hyperlipemia, hypercholesteremia, hyperglycemia, osteoporosis, obesity, impaired glucose tolerance, atherosclerosis, leptin resistance, insulin resistance, or diseases in which insulin resistance is the underlying pathophysiological mechanism comprising administering to a patient in need thereof an effective amount of a compound of formula (I) as defined in claim 1 or a compound as claimed in claim 7 or a pharmaceutical composition according to claim 8 or 9 in combination/concomittant with a HMG CoA reductase inhibitors, fibrates, nicotinic acid, cholestyramnine, colestipol or probucol or their combination within such a period so as to act synergistically.

17. A method according to claim 16, wherein the disease is type II diabetes, impaired glucose tolerance, dyslipidemia, disorders related to Syndrome X such as hypertension, obesity, insulin resistance, coronary artery disease and other cardiovascular disorders; certain renal diseases including glomerulonepliritis, glomerulosclerosis, nephrotic syndrome, hypertensive nephrosclerosis, retinopathy, nepliropathy, disorders related to endothelial cell activation, psoriasis, polycystic ovarian syndrome (PCOS), dementia, diabetic complications, osteoporosis, inflammatory bowel diseases, myotonic dystrophy, pancreatitis, arteriosclerosis, xanthoma, eating disorders, cancer or as inflammatory agents.

18. A method according to claim 16, for the treatment and/or prophylaxis of disorders related to Syndrome X, which comprises administering to a patient in need thereof an agonist of PPARα and/or PPARγ of formula (I) as claimed in claim 1 or a compound as claimed in claim 7 or a pharmaceutical composition according to claim 8 or 9 and a HMG CoA reductase inhibitors, fibrates, nicotinic acid, cholestyramine, colestipol or probucol or their combination within such a period as to act synergistically.

19. A method of reducing plasma glucose, triglycerides, total cholesterol, LDL, VLDL or free fatty acids or increasing HDL in the plasma, which comprises administering a compound of formula (I) claimed in claim 1 or a compound as claimed in claim 7 or a pharmaceutical composition according to claim 8 or 9, in combination/concomittant with a HMG CoA reductase inhibitor, fibrates, nicotinic acid, cholestyramine, colestipol or probucol which may be administered together or within such a period as to act synergistically together to a patient in need thereof.

20. The process as claimed in claim 5, wherein the alcohol is selected from ethanol, methanol, isopropanol, butanol or a mixture thereof.

21. The process as claimed in claim 5, wherein the ketone is selected from acetone, diethyl ketone, methyl ethyl ketone or mixture thereof.

22. The process as claimed in claim 5, wherein the ether is selected from diethyl ether, ether, tetrahydrofuran, dioxane, dibutyl ether or a mixture thereof.
